(19) United States
(12) Patent Application Publication
(10) Pub. No.: US 2012/0275952 A1
(43) Pub. Date: Nov. 1, 2012

(54) Method for Reducing the Concentration of Disinfectant, Decontamination Apparatuses and Systems and Related Methods of Employing the Same

(76) Inventors: Robert Lukasik, Lake Elsinore, CA (US); Szu-Min Lin, Irvine, CA (US); Robert C. Platt, JR., Temecula, CA (US); Todd Morrison, Dana Point, CA (US)

(21) Appl. No.: 13/098,386

(22) Filed: Apr. 29, 2011

(51) Int. Cl. A61L 2/18

(52) U.S. Cl. 422/28; 422/292; 422/291

(57) ABSTRACT

A method of reducing the concentration of disinfectant in an environment having a concentration of disinfectant of about 500 ppm or less for a period of time to a lower concentration is disclosed. The method comprises the steps of dehumidifying the environment for the period of time to remove disinfectant, and humidifying the environment with moisture during the period of time to maintain a difference between a lower relative humidity and an upper relative humidity of the environment ranging from about 20 to 50%. A system for decontaminating an environment is also disclosed, comprising a source of disinfectant, a source of moisture, one or more spray generators, and a dehumidifier.

### FIELD

The present disclosure relates generally to a method for reducing the concentration of disinfectant in an environment, decontamination apparatuses and systems, and related methods for decontaminating an environment.

### BACKGROUND

Enclosures and other environments, such as hospital and hotel rooms, tend to become contaminated with a wide variety of microbial contaminants, including bacteria, molds, fungi, yeasts, and the like. Some microbial contaminants are airborne and enter a room through doorways, windows, and/or ventilation systems. Other microbial contaminants are carried into the environment, such as on clothing, by occupants entering the room, and are transferred to surfaces or articles within the room via contact. These microorganisms are often able to survive in or on various surfaces in the room, such as carpets, drapes, wallpaper, furniture, countertops, and the like, or various articles positioned on the surfaces, and tend to be very difficult to eradicate.

In addition, environments may become contaminated with a variety of non-microbial contaminants, such as tobacco smoke, body perfume, and medicinal odors. These contaminants are, arguably, equally difficult to eradicate.

In the case of environments such as hospital and hotel rooms where the occupancy of a room changes frequently, it is desirable to ensure that both microbial and non-microbial decontaminants that are present in the room do not lead to contamination of a subsequent occupant.

Decontamination is a well known method of reducing or eradicating the microbial and non-microbial contaminants from an environment. Conventional decontamination processes typically involve one or more decontamination steps so that an environment is first decontaminated to an acceptable degree by introduction of disinfectant, followed by a series of numerous and lengthy steps to remove the disinfectant to an acceptable level. Of those steps, the removal of the disinfectant from the environment to a lower concentration, particularly removal of a disinfectant such as hydrogen peroxide to an acceptable level of 1 ppm or less, is a significant challenge when designing or developing an effective and efficient decontamination process. The decontamination process requires an environment to remain out of service during most or all of the process. Therefore, a lengthy decontamination process, specifically a lengthy disinfectant removal process, particularly when the environment is a hotel or hospital room, for example, results in significant downtime and loss of revenue while the room is unoccupied.

Accordingly, it would be advantageous to provide an alternative method to remove disinfectant from an environment, such that the disinfectant is removed rapidly so that the environment can be reoccupied quickly.

### SUMMARY

In one embodiment, the present disclosure provides a method of reducing the concentration of disinfectant in an environment having a concentration of disinfectant of about 500 ppm or less for a period of time to a lower concentration. The method comprises the steps of dehumidifying the environment during the period of time, and humidifying the environment continuously or intermittently with moisture during the period of time to maintain a difference of about 20 to 50% between a lower relative humidity and an upper relative humidity of the environment.

An additional aspect according to the present disclosure is directed to a decontamination system for decontaminating an environment, comprising a source of disinfectant, a source of moisture, one or more spray generators, and a dehumidifier. The spray generator is in fluid communication with at least one or both of the source of disinfectant and/or moisture, and is configured to release at least one of the disinfectant and moisture to the environment in the form of at least one of a mist or a vapor. The dehumidifier is configured to remove disinfectant and moisture from the environment.

It is understood that the invention disclosed and described herein is not limited to the embodiments disclosed in this Summary.

### BRIEF DESCRIPTION OF THE DRAWINGS

The characteristics of various non-limiting embodiments disclosed and described herein may be better understood by reference to the accompanying figures, in which:

FIG. 1 is a front perspective view of a decontamination apparatus of one non-limiting embodiment of the present disclosure;

FIG. 2 is a rear perspective view of a decontamination apparatus of one non-limiting embodiment of the present disclosure;

FIG. 3 is a front plan view of a decontamination apparatus of one non-limiting embodiment of the present disclosure with the front door of the apparatus in an open position;

FIG. 4 is a front plan view of a decontamination apparatus of one non-limiting embodiment of the present disclosure;

FIG. 5 is a graph of showing a method of reducing the concentration of disinfectant in an environment.

### DETAILED DESCRIPTION

In the present disclosure, other than where otherwise indicated, all numerical parameters are to be understood as being prefaced and modified in all instances by the term “about”, in which the numerical parameters possess the inherent variability characteristic of the underlying measurement techniques used to determine the numerical value of the parameter. At the very least, and not as an attempt to limit the application of the doctrine of equivalents to the scope of the claims, each numerical parameter described in the present description should at least be construed in light of the number of reported significant digits and by applying ordinary rounding techniques.

Also, any numerical range recited herein is intended to include all sub-ranges subsumed within the recited range. For example, a range of “1 to 10” is intended to include all sub-ranges between (and including) the recited minimum value of 1 and the recited maximum value of 10, that is, having a minimum value equal to or greater than 1 and a maximum value equal to or less than 10. Any maximum numerical limitation recited herein is intended to include all lower numerical limitations subsumed therein and any minimum numerical limitation recited herein is intended to include all higher numerical limitations subsumed therein. Accordingly, Applicant reserves the right to amend the present disclosure, including the claims, to expressly recite any sub-ranges subsumed within the ranges expressly recited herein. All such ranges are intended to be inherently disclosed herein such that amending to expressly recite any such sub-ranges would comply with the requirements of 35 U.S.C. §112, first paragraph, and 35 U.S.C. §132(a).

Any patent, publication, or other disclosure material that is said to be incorporated by reference herein, is incorporated herein in its entirety unless otherwise indicated, but only to the extent that the incorporated material does not conflict with existing definitions, statements, or other disclosure material expressly set forth in this description.

The term “decontamination” means the reduction of microorganisms to an acceptable level, not necessarily zero, and includes, but is not limited to, sanitization, disinfection and sterilization. For example, decontamination may also include the inactivation of prions, protozoal oocysts, bacterial endospores, mycobacteria, viruses, fungal spores, vegetative bacteria, and mycoplasmas; sanitization may refer to the reduction of microorganisms to levels considered safe from a public health viewpoint and typically requires less than a five log reduction of microorganisms; disinfection may refer to the reduction of disease-causing organisms on inanimate surfaces and typically requires at least a five log reduction of microorganisms; and sterilization may refer to the destruction of all microbial life, including spores, and typically requires at least a six log reduction of microorganisms.

The term “environment” means an open area, a contained area of gas or air, a closed area, a room, an isolator, an enclosure, or any suitable space, place, and/or area that may require decontamination. The term “environment” also comprises the surfaces, equipment, devices, beds, tables, and/or any other articles in the space, place, and/or area. Depending on the concentration of germicidal chemicals and applications, the term “environment” may also comprise poultry, and/or animals within the space, place, and/or area. In certain embodiments, the environment may be a room or “enclosure.” In certain other embodiments, the environment is a room of, for example, 25 to 100 m³. The enclosures may comprise windows and doors and may be furnished or unfurnished.

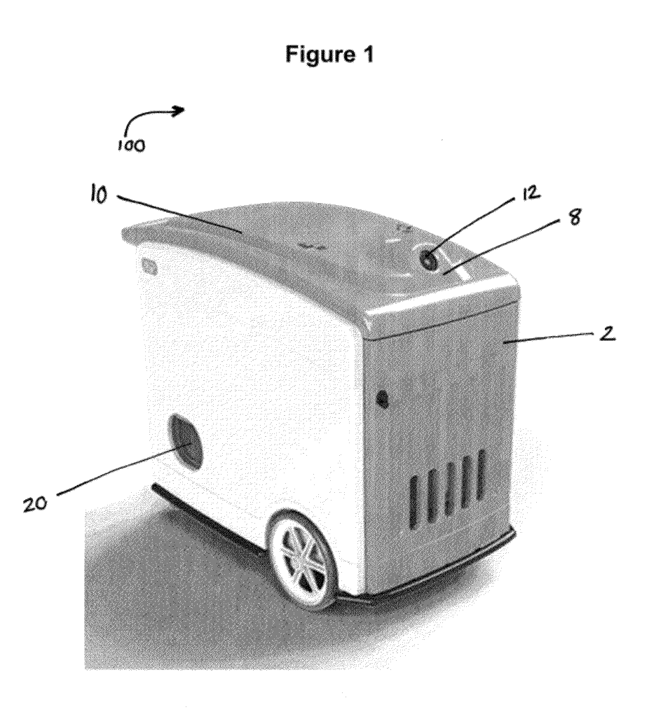

Referring now to FIG. 1-4, the present disclosure provides a decontamination apparatus 10 for sterilizing an environment 100, comprising a housing 2 comprising a source of disinfectant 4, a source of moisture 16, a spray generator 8 in fluid communication with the source of disinfectant 4 and the source of moisture 16 and configured to release the disinfectant and the moisture to the environment 100, and a dehumidifier 18 configured to remove the residual disinfectant and moisture from the environment 100.

As used herein, “a source of disinfectant” refers to a supply of disinfectant that is in fluid communication with the spray generator 8 of the decontamination apparatus 10. In one embodiment, and as illustrated, the source of disinfectant 4 may be a container or tank for retaining an amount of disinfectant in reserve. As used herein, “disinfectant” refers to various decontamination solutions known to those of ordinary skill in the art. The disinfectant may be comprised of a single or multiple component decontamination liquid or solution, such as electrolyzed water, miscible solutions of water and alcohols, biocides, such as hydrogen peroxide, organic compounds, peracetic acid, performic acid, other peracid chemical, acetic acid, ethoxylated additives (surfactants), ions, such as silver ions, ozonized liquid, chlorine compounds, hypochlorite, quaternary ammonium compounds, and mixtures thereof, oils and their blends, and combinations of any of the forgoing. Preferably, the disinfectant is an aqueous solution comprising hydrogen peroxide in a concentration ranging from less than about 40%, less than about 20%, less than about 10%, or about 5 to 8%. Although only one source of disinfectant 4 is illustrated, in certain non-limiting embodiments, it is contemplated that two or more sources of disinfectant may be employed, such as, for example, when the decontamination apparatus 10 is configured for use in very large environments, or if one or more reserve or back-up disinfectant sources are desired for convenience to the operator. The two or more sources of disinfectant may each contain the same or different disinfectants.

As used herein, a “source of moisture” refers to any container or device that retains or employs moisture to maintain the moisture content in an environment or, more typically, to increase the moisture content of an environment from a first humidity level to a second humidity level greater than the first, measured as absolute or relative humidity. The term “moisture” means any composition that includes a portion of free water that when added to an environment increases the absolute or relative humidity in the environment. Moisture may include water, in its various forms, or other compositions that include a mixture of water and various other compositions, including, for example, minor amounts of disinfectant. In certain embodiments, the source of moisture is comprised of water that is substantially free of disinfectant. In a preferred embodiment, the water is sterile. Absolute humidity is the number of pounds of water vapor associated with one pound (0.5 kg) of dry air, also just called humidity. Absolute units can include, for example, dew point or grains of water per pound of dry air. Relative humidity is the ratio, usually expressed as a percentage, of the partial pressure of water vapor in the atmosphere to the vapor pressure of water at the prevailing temperature. Relative humidity essentially describes the degree of saturation of the air.

The disinfectant may be dispensed from the decontamination apparatus 10 in the form of a mist and/or vapor. When the disinfectant is dispersed in the form of a mist, it is contemplated that some portion of the mist may vaporize or evaporate prior to or upon exiting the decontamination apparatus 10 to form a vapor. The term “mist” means a substance that is comprised of small droplets of liquid. Depending on the size and density of the small droplets of liquid, mist is generally visible to the naked eye. The term “vapor” means a gas that is comprised of free molecules. Vapor is produced from the evaporation of a mist or liquid. For the sake of clarity, in certain non-limiting embodiments, the apparatus and method set forth herein will be described in the form of the release of a “spray” or “mist” into the environment 100, although one of ordinary skill in the art will understand that vapor may be all, or at least a portion, of the stream exiting the apparatus 10 into the environment 100.

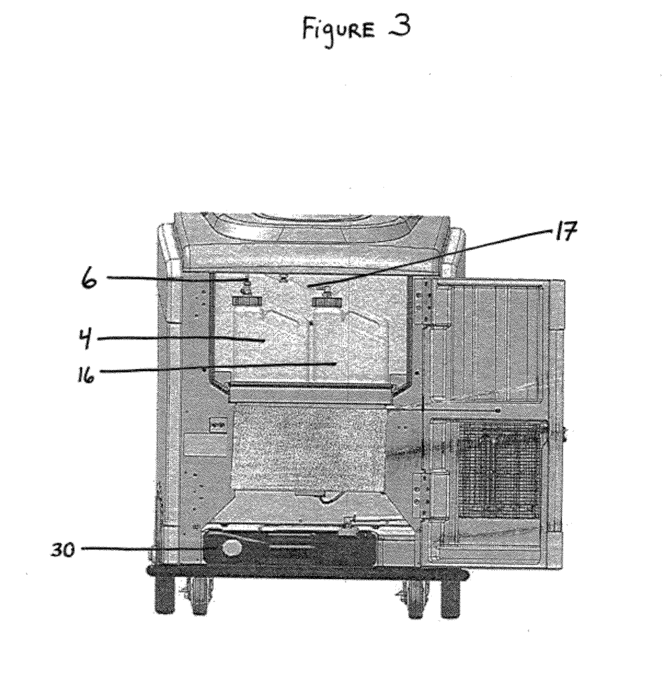

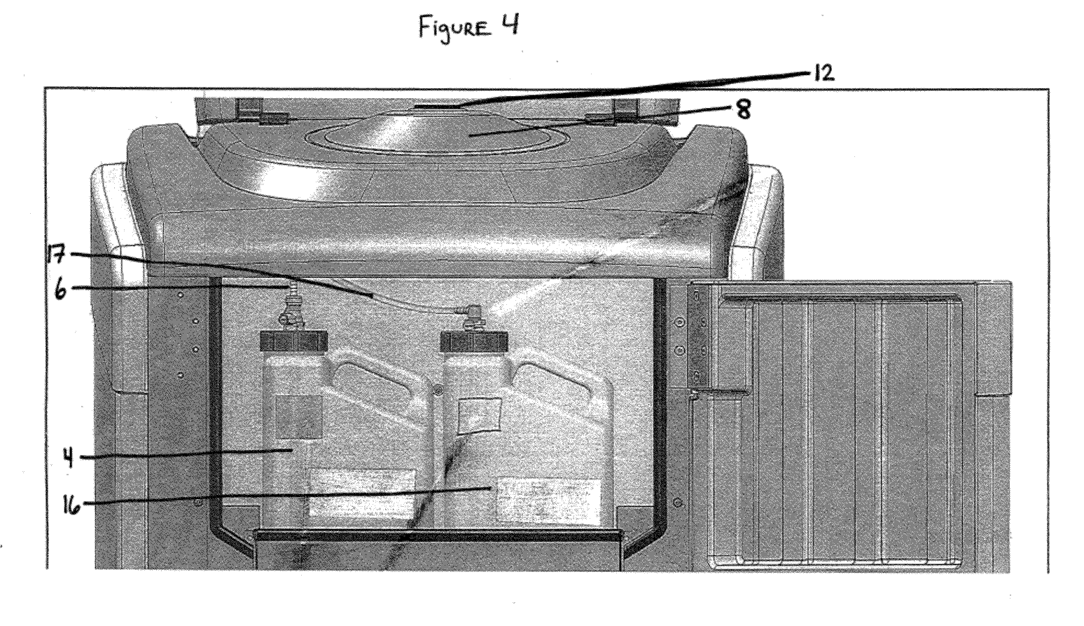

Referring to FIGS. 3-4, the source of disinfectant 4 may be in fluid communication via conduit 6 with a droplet or spray generator 8 having an outlet 12, such as a spray nozzle, for dispensing disinfectant into the environment 100. The spray generator 8 may be any conventional mist or liquid droplet generating apparatus known to those of skill in the art. In various embodiments, the warm, dried air from the environment may be channeled through the spray generator. In various embodiments, the spray generator 8 may generate a fine mist of less than about 1-20 micron, about 1-10 micron, about 1-5 micron, or about 5-10 micron diameter mist droplets. In one embodiment, the mist may be mono-dispense. In various embodiments, a commercially available spray generator, such as Fogmaster by Fogmaster Corporation, Deerfield Beach, Fla., for example, may be used to generate the mist. In various embodiments, the spray generator 8 may comprise an ultrasound humidifier or any other suitable spray or mist generator known to those of skill in the art. In certain embodiments, turbulent mixing may be employed wherein droplets of liquid are pulled into the airstream and sheared into smaller droplets by the turbulent air flow that collide with other droplets.

Alternatively, the spray generator 8 functions such that a pressure differential is created within the device to pull disinfectant from the source of disinfectant 4 and through the conduit 6. Owing to the fact that the first conduit 6 is in fluid communication with the spray generator 8, the disinfectant may be pulled through the first conduit 6 when the spray is exiting from the outlet 12, moving as result of the vacuum created by the movement of the spray into the environment 100. This pressure differential provides the force necessary to dispense the spray out of the outlet 12 and into the environment 100. It is contemplated that a disinfectant movement device (not shown) may also be employed to assist in the transfer of disinfectant to the spray generator 8. The disinfectant movement device may be a pump, fan, blower, and/or other suitable device configured to assist in the movement of disinfectant from the source of disinfectant 4 to the spray generator 8. The various components may be positioned within or on housing 2.

The quantity of mist produced by the spray generator 8 of the decontamination apparatus 10 is easily scalable for any environment by merely operating the decontamination apparatus 10 for a longer period of time, as the mist may be consistently produced as long as there is a source of disinfectant.

Referring again to FIG. 1-4, the decontamination apparatus 10 may also comprise at least one source of moisture 16 incorporated into the housing 2 of the decontamination apparatus 10 and configured to humidify the environment 100 with moisture. In certain embodiments, the source of moisture 16 may be in the form of a container or tank comprising a conduit 17 that supplies water to the spray generator 8 that dispels the moisture to the environment. As illustrated in FIGS. 3-4, the source of moisture 16 may be positioned within the housing 2, such as at a position adjacent the source of disinfectant 4.

In certain non-limiting embodiments, the decontamination apparatus 10 may include more than one spray generator (not shown). For example, in an embodiment wherein the decontamination apparatus 10 includes two spray generators, one disinfectant conduit from the source of disinfectant may be in fluid communication with the first spray generator and a separate moisture conduit from a source of moisture may be in fluid communication with the second spray generator. In certain other embodiments, the decontamination apparatus 10 may include more than two spray generators and comprise one or more disinfectant conduits from the at least one source of disinfectant and/or one or more moisture conduits from the at least one source of moisture to the desired number of spray generators.

As best illustrated in FIGS. 1-4, the decontamination apparatus 10 employs a single spray generator 8. In this embodiment, the source of disinfectant 4 and source of moisture 16 may be positioned in fluid communication with the spray generator 8 via disinfectant conduit 6, moisture conduit 17, wye, and the single conduit. In certain embodiments, all conduits including conduits 6 and 17 and wye are made of tubing that is bendable and compressible for ease of operation. In certain embodiments, the wye may be a triangular junction that enables the disinfectant conduit 6 and the moisture conduit 17 to supply their respective feeds through a single conduit that is in fluid communication with spray generator 8. The wye may receive disinfectant from conduit 6 and/or moisture from conduit 17 and allows the disinfectant and/or moisture feeds to pass through conduit 7 to the spray generator 8. The wye allows both disinfectant and moisture to be sprayed separately or simultaneously from a single spray generator 8. Although, multiple spray generators may be employed, as described herein, such that, for example, disinfectant and mist may be separately sprayed from separate spray generators, the wye allows for reduced component parts and cost. In certain embodiments, the source of moisture may be filtered through a filter or may be supplied in a sterile or disinfected state.

In certain non-limiting embodiments, moisture conduit 17 that connects the source of moisture 16 to the wye may be positioned lower than the disinfectant conduit 6 that connects the source of disinfectant 4 to the wye. Moisture conduit 17 may be advantageously positioned lower to allow the residual disinfectant that remains after spraying from the spray generator to drain down the water tube and into the source of moisture 16 whenever the spray generator is turned off. As illustrated in FIGS. 3, in certain embodiments, the decontamination apparatus 10 may comprise a drain tank 30. The drain tank 30 may be positioned at the lower most portion of the decontamination apparatus 10, as illustrated. The drain tank 30 may be positioned to capture residual moisture and other by-products that may result from the operation of the decontamination apparatus 10, such as excess disinfectant or other solutions from the decontamination operation or water from the dehumidification process.

In certain other embodiments, the source of disinfectant 4 and source of moisture 16 may include electronic tags to store information, such as product information, lot numbers, expiration dates, volumes, and the like. The information stored in the tags may be read by an RFID reader. The RFID reader may be in periodic communication with the tags to update information, fill volumes, and check for expiration dates. The communication between the RFID tags on a source of disinfectant 4 and source of moisture 16 allow the operator of the decontamination apparatus 10 to be notified of, for example, the remaining amount of disinfectant or moisture in the respective containers. In certain non-limiting embodiments, at least one RFID reader may be located adjacent to the source of disinfectant 4 and source of moisture 16.

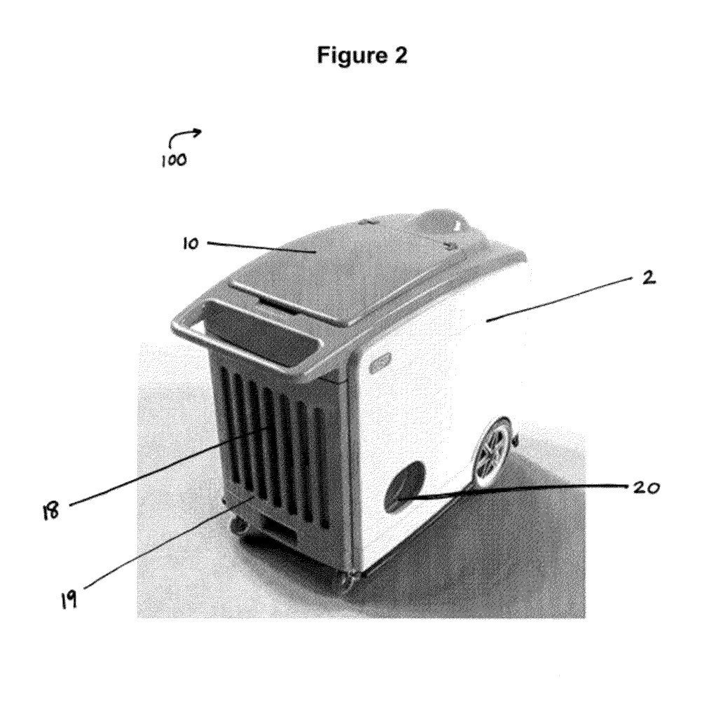

The decontamination apparatus 10 may also comprise at least one dehumidifier 18 configured to remove disinfectant and moisture from the environment 100. As illustrated in FIG. 2, the dehumidifier 18 may be positioned within the housing 2, such as at the rear portion of the decontamination apparatus 10, as illustrated. When incorporated in the housing 2, the decontamination apparatus 10 may further comprise a dehumidifier intake 19 that allows air having moisture and/or disinfectant therein from the environment 100 to be drawn into the dehumidifier 18. Either or both sides of the decontamination apparatus 10 may further comprise a dehumidifier exhaust 20 that expels at least a portion of the a treated air from the dehumidifier 18 back to the environment 100. The dehumidifier 18 may be any conventional dehumidifier known to those of ordinary skill in the art, such as the condensing type dehumidifier Santa Fe Max Dry Dual XT with 150 pint per day (ppd) moisture removal capacity, manufactured by Therma-Stor LLC, located in Madison, Wis., preferably one that removes moisture at a rate greater than 140 ppd @ AHAM.

In certain non-limiting embodiments, the decontamination apparatus 10 may further comprise a scrubber (not shown). The scrubber may be any wet or dry scrubber-type air pollution control device that can be used to remove particulates and/or gases from the environment 100. When a scrubber is employed, various catalysts, such as a palladium catalyst, may be used to remove residual gases or additional residual disinfectant, such as hydrogen peroxide, from the environment 100.

In certain non-limiting embodiments, the decontamination apparatus 10 may further comprise various sensing devices that aid in monitoring the operation of the various components of the decontamination apparatus 10 during operation. For example, the decontamination apparatus 10 may comprise, for example one or more humidity sensors, disinfectant sensors, and/or level sensors (not shown). The disinfectant sensor may be a hydrogen peroxide sensor when hydrogen peroxide is employed as the disinfectant to measure the concentration of the disinfectant in the environment 100. The humidity sensor may be positioned to monitor the relative humidity of the environment 100 either in the housing 2 or affixed to an object in the environment. The level sensor may be a capacitive sensor that detects the level of a solution by sensing the solution's change in electrical capacity with respect to the height of the solution. The capacitive level sensor may be employed, for example, to notify an operator of the decontamination apparatus 10 that the draining tank 30 is full and requires that liquid be drained from the draining tank 30. In certain embodiments, the decontamination apparatus 10 will shut-off if the capacitive level sensor is signaling that the draining tank 30 is full.

In certain non-limiting embodiments, the decontamination apparatus 10 may comprise a remote control (not shown) for controlling various aspects of the operation of the decontamination device 10 as known to those of ordinary skill in the art.

The present disclosure also provides a decontamination system for decontaminating environment 100, comprising a source of disinfectant 4, a source of moisture 16, one or more spray generators 8, and a dehumidifier 18. It is contemplated that the decontamination system comprises components and operations similar to those described herein, wherein at least one component, such as, for example, the source of moisture 16, the dehumidifier 18, is positioned outside of the housing 2 but within the environment 100. In these embodiments, it is contemplated that the one or more components separate from the housing 2 may or may not be in fluid communication with the decontamination apparatus 10. Furthermore, the operation of these separate components may be employed in concert with the decontamination apparatus or may be independent and separately controllable therefrom. For example, the source of moisture may be a humidifier such as Crane Model #EE-3186 humidifier, having the capacity to deliver up to about 2.1 gallons of moisture per day, by Crane USA, Inc., that is positioned external and separate from decontamination apparatus 10.

During a decontamination process, a disinfectant may be introduced into the environment one or more times, followed by a method of reducing the concentration of disinfectant in the environment. Various embodiments disclosed herein are directed to a method of reducing the concentration of disinfectant in an environment having a concentration of disinfectant of about 500 ppm or less, about 100 to 500, or about 10 to 100 ppm, in a period of time to a lower concentration by removal of the disinfectant from the environment using dehumidification. In certain embodiments, a concentration of disinfectant of about 50 ppm or less, or about 10 to 50 ppm, may be reduced in a period of time to a lower concentration. In certain other embodiments, a concentration of disinfectant of about 10 ppm or less, or about 2 to 10 ppm, may be reduced in a period of time to a lower concentration. As used herein, the term “concentration of disinfectant in an environment” excludes the disinfectant residing within the decontamination apparatus 10.

In certain embodiments, the environment may have a temperature of about 55 to 95° F. or about 68 to 80° F. when disinfectant is introduced into the environment.

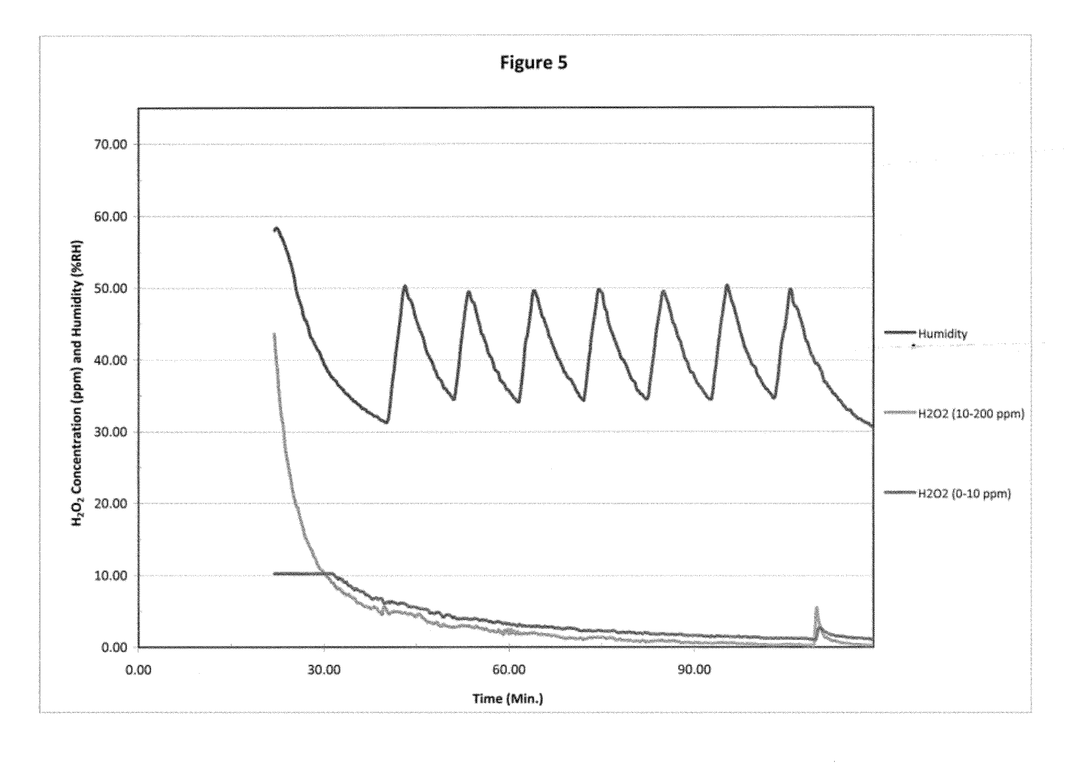

In certain embodiments, the method of reducing the concentration of disinfectant in an environment may remove hydrogen peroxide using dehumidification from the environment to a lower concentration, as exemplified in FIG. 5. For example, the method of reducing the concentration of disinfectant in an environment may reduce the disinfectant concentration to about 10 ppm, 5 ppm or 3 ppm or less. When hydrogen peroxide is utilized as the disinfectant in a decontamination process, it may be desirable to reduce the concentration of disinfectant in the environment to a concentration that is deemed acceptable by governmental regulations before humans can re-enter the environment. As an example, a hydrogen peroxide concentration that has been deemed to be safe for humans is about 1 ppm or less. However, this level may be higher in certain countries and as such the acceptable level will vary according to the governmental regulations of different countries.

During the period of time when disinfectant is removed from the environment using the dehumidifier, dehumidification of the environment may occur continuously until the disinfectant concentration reaches the lower level, while humidification of the environment may occur intermittently or continuously depending upon the output of the humidifier during the same period of time. Alternatively, during the period of time when disinfectant is removed from the environment using the dehumidifier, dehumidification of the environment may occur intermittently until the disinfectant concentration reaches the lower level, while humidification of the environment may occur intermittently or continuously depending upon the output of the humidifier during the same period of time. Alternatively, during the period of time when disinfectant is removed from the environment using the dehumidifier, both dehumidification and humidification may be operated intermittently, wherein at any given moment the dehumidification and humidification may be on simultaneously, or one of dehumidification and humidification may be on and the other off, or dehumidification and humidification may both be off for a time.

As used herein in conjunction with dehumidification and humidification, the term “intermittently” refers to cycling dehumidification and/or humidification on and off at least once at evenly spaced or variable intervals of time. The duty cycle, defined herein as the ratio of the time on to the total time of the period under consideration, for dehumidification may be the same or different than the duty cycle for humidification, depending upon the relative removal rate and output of the dehumidification and humidification units. Additionally, dehumidification and humidification may be cycled in phase or out of phase with one another.

Without wishing to be bound by theory, possible mechanisms for the more efficient removal of hydrogen peroxide from the environment by continuous or intermittent humidification with dehumidification as described above are (1) that any hydrogen peroxide that may be present in the vapor phase dissolves in the droplets of moisture delivered by the humidifier; (2) that any hydrogen peroxide that may be present in the liquid phase as droplets may collide, and coalesce with, and be diluted by, the droplets of moisture delivered by the humidifier; (3) that any hydrogen peroxide that may be present in the vapor phase may condense out of the atmosphere due to the saturation of the atmosphere by moisture delivered by the humidifier, and be subject to coalescence and dilution as above or may fall out on surfaces where during the dehumidification portion of the process it may be re-evaporated and subject to dissolving in the moisture as above; and/or (4) that due to the higher molecular weight and higher vapor pressure as compared to water, that the hydrogen peroxide will condense before water as the relative humidity increases due to the moisture delivered by the humidifier.

In certain embodiments, humidification of the environment with a source of moisture, during the period disinfectant is removed from the environment using dehumidification, maintains a difference of about 20 to 50% between a lower relative humidity and an upper relative humidity of the environment, for example humidification may maintain a difference of about 50% between a lower relative humidity and an upper relative humidity of the environment, for example but not limited to a relative humidity of the environment that ranges from about 30 (lower relative humidity) and 80% (upper relative humidity). Alternatively, humidification may maintain a difference of about 40%, for example but not limited to a relative humidity of the environment that ranges from about 40 and 80%, a difference of about 30%, for example but not limited to a relative humidity of the environment that ranges from about 25 to 55%, a difference of about 25%, for example but not limited to a relative humidity of the environment that ranges from about 30 to 55%, or a difference of about 20%, for example but not limited to a relative humidity of the environment that ranges from about 25 to 45%.

In certain embodiments, humidification of the environment with a source of moisture during the period disinfectant is removed from the environment maintains the relative humidity in the environment between about 25 to 80%, about 25 to 55%, about 30 to 55%, or about 25 to 45%.

During the period of time when disinfectant is removed from the environment, dehumidification may be turned on first to reduce the concentration of disinfectant in the environment. Thereafter but during the same period of time, when the relative humidity of the environment reaches about 25%, humidification may be turned on for example to maintain the relative humidity at about 25 to 55%.

In certain embodiments, the combination of dehumidification and humidification of the environment with moisture to maintain the relative humidity of the environment between about 25 to 80% allows disinfectant to be removed until the concentration of the disinfectant in the environment reaches acceptable lower concentration level, for example about 10.0 ppm or less, about 5.0 ppm or less, about 3.0 ppm or less, or about 1.0 ppm or less, in a shorter period of time compared to the use of dehumidification alone. In certain embodiments, the step of humidifying the environment during the period of time may introduce moisture at a rate less than 300 ml/min, or alternatively ranging from about 10 to 80 ml/min, about 40 to 70 ml/min, or at a rate that will humidify a particular room size to a desired humidity within a desired period of time.

In certain embodiments, dehumidification and humidification may be automated or manual. In certain embodiments, dehumidification and humidification may be automatically controlled through the use of sensing devices and controllers, such that manual operation is unnecessary.

In certain non-limiting embodiments, the method of reducing the concentration of disinfectant may further comprise at least one scrubbing step using a scrubber for example that comprises a palladium catalyst. Although the use of a scrubber may reduce the time required to reduce the concentration of disinfectant from the environment to a lower concentration, i.e., to an acceptable level for human re-entry into the environment, the cost of a scrubber, particularly a catalytic scrubber, significantly increases the cost of the decontamination apparatus. Therefore, even though a scrubbing step may be utilized with the method of reducing the concentration of disinfectant from the environment according to the present invention, it may be more desirable to reduce costs and to exclude the scrubber from decontamination apparatus described herein.

In certain embodiments, the methods of decontaminating the environment may comprise introducing a disinfectant comprising hydrogen peroxide into the environment followed by the method of reducing the concentration of the disinfectant described above, to reduce the level of clinical microorganisms by 3, 4, 5 or 6 logs.

The method of decontaminating an environment may comprise one or more introductions of disinfectant to achieve for example, either a concentration or amount of disinfectant in the room, or a relative humidity, or until the required level of decontamination is met. For example, disinfectant may be introduced into the environment during an initial system check step to ensure that the decontamination system is operational, i.e., the spray generator is functioning, and/or a disinfectant introduction step, such as first disinfectant injection, second disinfectant injection, etc.

In certain embodiments, the method of decontaminating an environment may comprise introducing hydrogen peroxide until a desired relative humidity, for example about 50 to 60%, is reached followed by a period of time to allow the disinfectant to dwell within the environment, and thereafter the method for reducing the concentration of the disinfectant in the environment may be performed. In certain other embodiments, the method of decontaminating the environment may comprise more than one cycle that includes introducing hydrogen peroxide until a desired relative humidity, for example about 70 to 80%, is reached, allowing the disinfectant to dwell in the environment and dehumidifying the environment to a relative humidity of 60 to 70% prior to the next repeated cycle, and thereafter the method for reducing the concentration of the disinfectant in the environment may be performed.

In certain non-limiting embodiments, disinfectant may be introduced while accompanied by the use of dehumidification at the same time or for overlapping periods of time to concentrate the disinfectant on the surfaces of the environment. While not wishing to be bound by theory, hydrogen peroxide, for example, having a higher molecular weight and lower vapor pressure will condense before water when the humidity is increased; and water having a lower molecular weight and higher vapor pressure will evaporate before hydrogen peroxide when the humidity is lowered with the net effect that intermittent humidification and dehumidification allows the concentration of hydrogen peroxide in the environment.

In certain non-limiting embodiments, the water in a water-based disinfectant may be used to verify that disinfectant is actually being delivered during the disinfectant introduction step, using a humidity sensor, thereby eliminating the need to use a separate disinfectant sensor.

The illustrative and non-limiting examples that follow are intended to further describe various non-limiting embodiments without restricting the scope of the embodiments. Persons having ordinary skill in the art will appreciate that variations of the Examples are possible within the scope of the invention as defined by the claims.

### EXAMPLE

A study was conducted to demonstrate that the time to remove disinfectant from the high level remaining after a room is disinfected to the low level acceptable for room re-entry may be dramatically reduced by intermittent humidification and dehumidification of a room. In this example a 43 m³ room at 40% RH was treated with disinfectant. The amount of water dispensed by the humidifier was determined by weighing the water tank before and after each test run. In Test cases 1 and 2, no water was dispensed into the room during the disinfectant removal portion. In Test cases 3 and 4 water was used and a dehumidifier and humidifier were running during the disinfectant removal portion. The time to reach a level of less than or equal to 1 ppm disinfectant was recorded. Test cases 1 and 2, where the dehumidifier was used without humidification, were terminated after 5.02 hours since the dehumidifier alone was ineffective in reducing the disinfectant to 1 ppm. In Test cases 3 and 4, less than or equal to 1 ppm was achieved in about one and one half hours, demonstrating that the concentration of disinfectant could be reduced to an acceptable level in a shorter period of time if water is dispensed into the room in combination with dehumidification.

TABLE I
Disinfectant Reduction Time Comparative Study of
Dehumidification Only vs. Dehumidification with Humidification

| Test Case | Total Water Weight (g) | Total Time to 1 ppm (hours) |
|---|---|---|
| 1 | 34.9 | 5.02 (canceled) |
| 2 | 11.3 | 5.02 (canceled) |
| 3 | 892.2 | 1.42 |
| 4 | 1411.2 | 1.48 |

This disclosure has been written with reference to various exemplary, illustrative, and non-limiting embodiments. However, it will be recognized by persons having ordinary skill in the art that various substitutions, modifications, or combinations of any of the disclosed embodiments (or portions thereof) may be made without departing from the scope of the invention. Thus, it is contemplated and understood that the present disclosure embraces additional embodiments not expressly set forth herein. Such embodiments may be obtained, for example, by combining, modifying, or reorganizing any of the disclosed steps, components, elements, features, aspects, characteristics, limitations, and the like, of the embodiments described herein. In this regard, Applicant reserves the right to amend the claims during prosecution to add features as variously described herein.

1. A method of reducing the concentration of disinfectant in an environment having a concentration of disinfectant of about 500 ppm or less for a period of time until the concentration of the disinfectant in the environment is reduced to a lower concentration, comprising the steps of:
dehumidifying the environment continuously or intermittently during the period of time to remove the disinfectant; and
humidifying the environment continuously or intermittently with moisture during the period of time to maintain a difference of about 20 to 50% between a lower relative humidity and an upper relative humidity of the environment.

2. The method of claim 1, wherein the environment has a concentration of about 100 ppm or less.

3. The method of claim 1, wherein the environment has a concentration of about 10 ppm or less.

4. The method of claim 1, wherein the step of humidifying the environment is performed until the lower concentration is about 10.0 ppm or less.

5. The method of claim 3, wherein the step of humidifying the environment is performed until the lower concentration is about 5.0 ppm or less.

6. The method of claim 3, wherein the step of humidifying the environment is performed until the lower concentration is about 3.0 ppm or less.

7. The method of claim 3, wherein the step of humidifying the environment is performed until the lower concentration is about 1.0 ppm or less.

8. The method of claim 1, wherein the step of humidifying the environment maintains the relative humidity in the environment between about 25 to 55%.

9. The method of claim 1, wherein the step of humidifying the environment maintains the relative humidity in the environment between about 30 to 55%.

10. The method of claim 1, wherein the step of humidifying the environment maintains the relative humidity in the environment between about 25 to 45%.

11. The method of claim 7, wherein the disinfectant is hydrogen peroxide.

12. The method of claim 11, wherein the moisture comprises water that is substantially free of disinfectant.

13. The method of claim 12, wherein the water is sterile.

14. A method of decontaminating an environment comprising the step of introducing a hydrogen peroxide disinfectant into the environment and reducing the concentration of disinfectant according to claim 1.

15. The method of decontaminating an environment of claim 14, wherein the hydrogen peroxide disinfectant is introduced into the environment during a first injection period, and further comprising the step of dehumidifying the environment during the first injection period to a relative humidity of about 40 to 60%.

16. The method of decontaminating an environment of claim 15, wherein the hydrogen peroxide disinfectant is introduced into the environment at a rate ranging from about 40 to 70 ml/min and the step of dehumidifying the environment during the first injection period removes moisture at a rate greater than 140 ppd @ AHAM.

17. The method of decontaminating an environment of claim 14, wherein the step of introducing hydrogen peroxide disinfectant is repeated one or more times.

18. The method of decontaminating an environment of claim 17, further comprising the step of dehumidifying the environment prior to repeating the step of introducing hydrogen peroxide disinfectant.

19. A decontamination system for decontaminating an environment, comprising:
a source of disinfectant;
a source of moisture;
one or more spray generators in fluid communication with at least one or both of the source of disinfectant and/or moisture, the spray generator configured to release at least one of the disinfectant and moisture to the environment in the form of at least one of a mist or a vapor; and
a dehumidifier configured to remove disinfectant and moisture from the environment.

20. The decontamination system of claim 19, further comprising a housing that comprises at least two of the source of disinfectant, the source of moisture, the one or more spray generators, and the dehumidifier.

21. The decontamination system of claim 19, further comprising a scrubber.

22. The decontamination system of claim 19, wherein the disinfectant is hydrogen peroxide.
